(19) United States
(12) Patent Application Publication
(10) Pub. No.: US 2007/0016056 A1
(43) Pub. Date: Jan. 18, 2007

(54) Methods and systems for providing control components in an ultrasound system

(76) Inventor: Scott Kerwin, Charlotte, NC (US)

Correspondence Address: THE SMALL PATENT LAW GROUP LLP, 611 OLIVE STREET, SUITE 1611, ST. LOUIS, MO, 63101, US

(21) Appl. No.: 11/182,128

(22) Filed: Jul. 15, 2005

(51) Int. Cl. A61B 8/14

(52) U.S. Cl. 600/459

(57) ABSTRACT

Methods and systems for providing control components in an ultrasound system are provided. An ultrasound probe and the ultrasound system includes a scanned portion for scanning an object and a connector for connecting the scanned portion to the ultrasound system. The ultrasound probe also includes at least one printed circuit board within at least one of the scanned portion and the connector, with the at least one printed circuit board having invented passive components.

### BACKGROUND OF THE INVENTION

This invention relates generally to ultrasound systems, and more particularly, to methods and systems for providing control components in an ultrasound system, especially in a probe of the ultrasound system.

Ultrasound systems typically include ultrasound scanning devices, such as, ultrasound probes having different control components and transducers that allow for performing various different ultrasound scans (e.g., different imaging of a volume or body). These ultrasound probes may include control components within different portions of the probe, including, for example, the probe handle and the probe connection member for connecting to an ultrasound system. These control components within the probe allow for controlling operation of the probe by an ultrasound system, for example, to operate in different modes, such as, amplitude mode (A-mode), brightness mode (B-1 mode), power Doppler mode, color imaging mode, among others.

Most ultrasound probes include control components provided as part of a printed circuit board, sometimes referred to as a printed wiring board. These control components are mounted on the printed circuit board and used when controlling the probe. For example, these control components may include discrete passive electrical components, such as, resistors, inductors, and capacitors surface mounted to the printed circuit board. Thus, the printed circuit boards are populated with these passive electrical components after the board is fabricated. Further, one or more discrete components are required for each transducer element within the probe, thereby requiring surface space on the printed circuit board. Thus, depending on the number of transducer elements in the ultrasound probe, the number of passive electrical components required to control the transducer elements and surface mounted on the printed circuit board, a large surface area on the printed circuit board may be needed for these components, which requires a larger housing, for example, for the probe or the probe connector.

Thus, current probe designs using control components surface mounted to printed circuit boards require space for each of the control components mounted to the printed circuit board. As the number of transducer elements increases, for example, when large arrays of transducer elements are implemented, the size of the probe and probe connector also must increase to accommodate the size of the printed circuit boards. This may result in ultrasound probes having larger than desired housings or casings.

### BRIEF DESCRIPTION OF THE INVENTION

In one embodiment, an ultrasound probe is provided. The ultrasound probe includes a scan portion for scanning an object, a connector for connecting the scan portion to an ultrasound system, and at least one printed circuit board within at least one of the scanned portion and the connector. The at least one printed circuit board includes embedded passive components.

In another embodiment, a method for controlling operation of an ultrasound probe is provided. The method includes imbedding passive electrical components in a printed circuit board for connection within an ultrasound probe and configuring the embedded passive electrical components to communicate with an ultrasound system for controlling the ultrasound probe.

### BRIEF DESCRIPTION OF THE DRAWINGS

FIG. 1 is a block diagram of an ultrasound system in accordance with an exemplary embodiment of the present invention.

FIG. 2 is a block diagram of an ultrasound system in accordance with another exemplary embodiment of the present invention.

FIG. 3 is a perspective view of an image of an object acquired by the systems of FIGS. 1 and 2 in accordance with an exemplary embodiment of the present invention.

FIG. 4 is a perspective view of a prior art ultrasound probe showing a printed circuit board therein.

FIG. 5 is a side elevation view of a prior art printed circuit board.

FIG. 6 is a side elevation view of a printed circuit board in accordance with an exemplary embodiment of the present invention.

FIG. 7 is a side elevation view of a printed circuit board in accordance with another exemplary embodiment of the present invention.

FIG. 8 is a schematic representation of an embedded inductor component in accordance with an exemplary embodiment of the present invention.

FIG. 9 is a schematic representation of an embedded inductor component in accordance with another exemplary embodiment of the present invention.

FIG. 10 is a schematic representation of a capacitor component in accordance with an exemplary embodiment of the present invention.

FIG. 11 is a schematic representation of a capacitor component in accordance with another exemplary embodiment of the present invention.

FIG. 12 is a schematic representation of a resistor component in accordance with an exemplary embodiment of the present invention.

FIG. 13 is a top plan view of a printed circuit board having embedded controlled components in accordance with an exemplary embodiment of the present invention.

FIG. 14 is a block diagram of an ultrasound system having a probe with a printed circuit board therein in accordance with an exemplary embodiment of the present invention.

### DETAILED DESCRIPTION OF THE INVENTION

Exemplary embodiments of ultrasound systems and methods for providing control components in ultrasound systems are described in detail below. In particular, a detailed description of exemplary ultrasound systems is first provided followed by a detailed description of various embodiments of methods and systems for providing control components in an ultrasound system. A technical effect of the various embodiments of the systems and methods described herein include at least one of improving the design of an ultrasound probe and reducing the size for constructing such ultrasound probes.

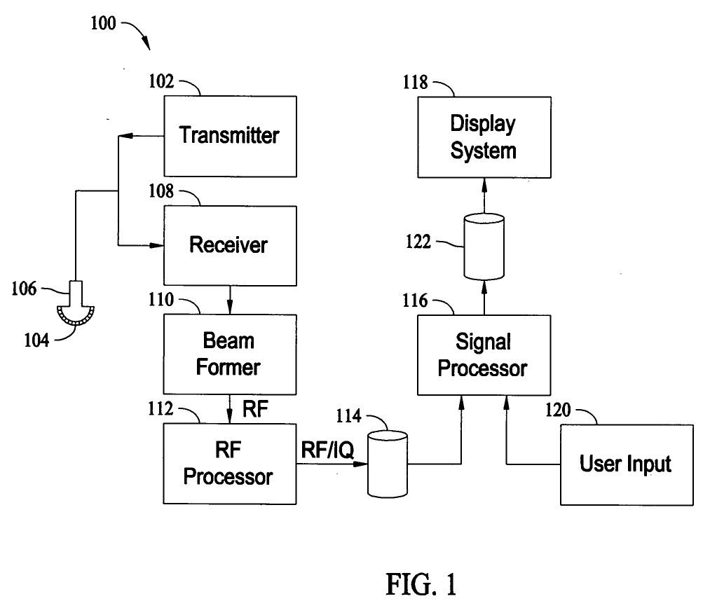

FIG. 1 illustrates a block diagram of an exemplary embodiment of an ultrasound system 100 that may be used, for example, to acquire and process ultrasonic images. The ultrasound system 100 includes a transmitter 102 that drives an array of elements 104 (e.g., piezoelectric crystals) within or formed as part of a transducer 106 to emit pulsed ultrasonic signals into a body or volume. A variety of geometries may be used and one or more transducers 106 may be provided as part of a probe (not shown). The pulsed ultrasonic signals are back-scattered from density interfaces and/or structures, for example, in a body, like blood cells or muscular tissue, to produce echoes that return to the elements 104. The echoes are received by a receiver 108 and provided to a beamformer 110. The beamformer performs beamforming on the received echoes and outputs an RF signal. The RF signal is then processed by an RF processor 112. The RF processor 112 may include a complex demodulator (not shown) that demodulates the RF signal to form IQ data pairs representative of the echo signals. The RF or IQ signal data then may be routed directly to an RF/IQ buffer 114 for storage (e.g., temporary storage).

The ultrasound system 100 also includes a signal processor 116 to process the acquired ultrasound information (i.e., RF signal data or IQ data pairs) and generate frames of ultrasound information for display on a display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 114 during a scanning session and processed in less than real-time in a live or off-line operation.

The ultrasound system 100 may continuously acquire ultrasound information at a frame rate that exceeds fifty frames per second, which is the approximate perception rate of the human eye. The acquired ultrasound information is displayed on the display system 118 at a slower frame-rate. An image buffer 122 may be included for storing processed frames of acquired ultrasound information that are not scheduled to be displayed immediately. In an exemplary embodiment, the image buffer 122 is of sufficient capacity to store at least several seconds of frames of ultrasound information. The frames of ultrasound information may be stored in a manner to facilitate retrieval thereof according to their order or time of acquisition. The image buffer 122 may comprise any known data storage medium.

A user input device 120 may be used to control operation of the ultrasound system 100. The user input device 120 may be any suitable device and/or user interface for receiving user inputs to control, for example, the type of scan or type of transducer to be used in a scan.

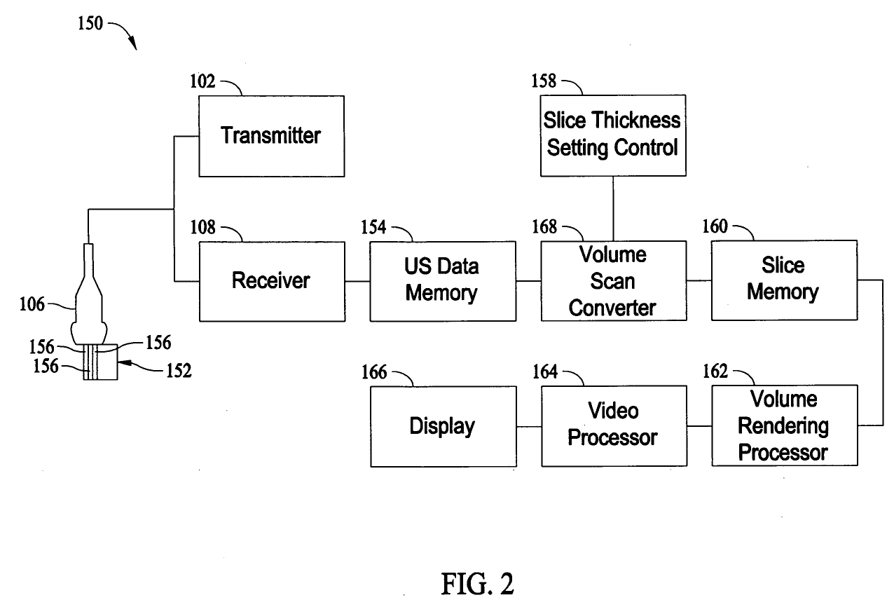

FIG. 2 illustrates a block diagram of another exemplary embodiment of an ultrasound system 150 that may be used, for example, to acquire and process ultrasonic images. The ultrasound system 150 includes the transducer 106 in communication with the transmitter 102 and receiver 108. The transducer 106 transmits ultrasonic pulses and receives echoes from structures inside a scanned ultrasound volume 152. A memory 154 stores ultrasound data from the receiver 108 derived from the scanned ultrasound volume 152. The scanned ultrasound volume 152 may be obtained by various techniques, including, for example, 3D scanning, real-time 3D imaging, volume scanning, scanning with transducers having positioning sensors, freehand scanning using a Voxel correlation technique, 2D scanning or scanning with a matrix of array transducers, among others.

The transducer 106 is moved, such as along a linear or arcuate path, while scanning a region of interest (ROI). At each linear or arcuate position, the transducer 106 obtains a plurality of scan planes 156. The scan planes 156 are collected for a thickness, such as from a group or set of adjacent scan planes 156. The scan planes 156 are stored in the memory 154, and then provided to a volume scan converter 168. In some exemplary embodiments, the transducer 106 may obtain lines instead of the scan planes 156, with the memory 154 storing lines obtained by the transducer 106 rather than the scan planes 156. The volume scan converter 168 receives a slice thickness setting from a slice thickness setting control 158, which identifies the thickness of a slice to be created from the scan planes 156. The volume scan converter 168 creates a data slice from multiple adjacent scan planes 156. The number of adjacent scan planes 156 that are obtained to form each data slice is dependent upon the thickness selected by the slice thickness setting control 158. The data slice is stored in a slice memory 160 and accessed by a volume rendering processor 162. The volume rendering processor 162 performs volume rendering upon the data slice. The output of the volume rendering processor 162 is provided to a video processor 164 that processes the volume rendered data slice for display on a display 166.

It should be noted that the position of each echo signal sample (Voxel) is defined in terms of geometrical accuracy (i.e., the distance from one Voxel to the next) and one or more ultrasonic responses (and derived values from the ultrasonic response). Suitable ultrasonic responses include gray scale values, color flow values, and angio or power Doppler information.

It should be noted that the ultrasound systems 100 and 150 may include additional or different components. For example, the ultrasound system 150 may include a user interface or user input 120 (shown in FIG. 1) to control the operation of the ultrasound system 150, including, to control the input of patient data, scan parameters, a change of scan mode, and the like.

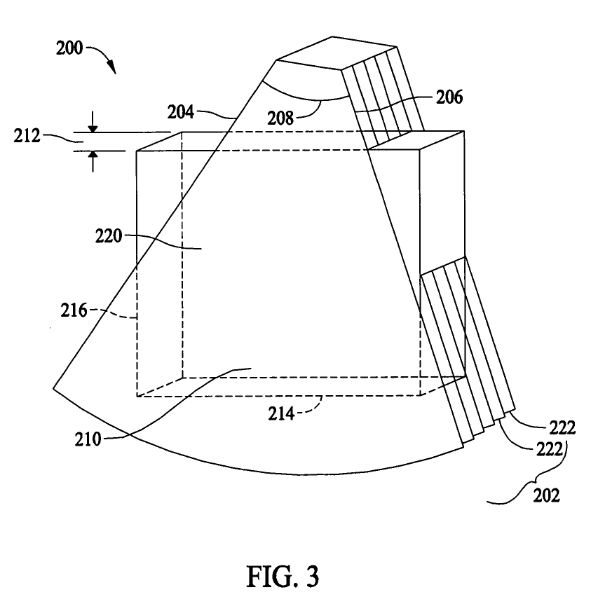

FIG. 3 illustrates an exemplary image of an object 200 that may be acquired by the ultrasound systems 100 and 150. The object 200 includes a volume 202 defined by a plurality of sector shaped cross-sections with radial borders 204 and 206 diverging from one another at an angle 208. The transducer 106 (shown in FIGS. 1 and 2) electronically focuses and directs ultrasound firings longitudinally to scan along adjacent scan lines in each scan plane 156 (shown in FIG. 2) and electronically or mechanically focuses and directs ultrasound firings laterally to scan adjacent scan planes 156. The scan planes 156 obtained by the transducer 106, and as illustrated in FIG. 1, are stored in the memory 154 and are scan converted from spherical to Cartesian coordinates by the volume scan converter 168. A volume comprising multiple scan planes 156 is output from the volume scan converter 168 and stored in the slice memory 160 as a rendering region 210. The rendering region 210 in the slice memory 160 is formed from multiple adjacent scan planes 156.

The rendering region 210 may be defined in size by an operator using a user interface or input to have a slice thickness 212, width 214 and height 216. The volume scan converter 168 (shown in FIG. 2) may be controlled by the slice thickness setting control 158 (shown in FIG. 2) to adjust the thickness parameter of the slice to form a rendering region 210 of the desired thickness. The rendering region 210 defines the portion of the scanned ultrasound volume 152 that is volume rendered. The volume rendering processor 162 accesses the slice memory 160 and renders along the slice thickness 212 of the rendering region 210.

Referring now to FIGS. 1 and 2, during operation, a slice having a pre-defined, substantially constant thickness (also referred to as the rendering region 210) is determined by the slice thickness setting control 158 and is processed in the volume scan converter 168. The echo data representing the rendering region 210 (shown in FIG. 3) may be stored in the slice memory 160. Predefined thicknesses between about 2 mm and about 20 mm are typical, however, thicknesses less than about 2 mm or greater than about 20 mm may also be suitable depending on the application and the size of the area to be scanned. The slice thickness setting control 158 may include a control member, such as a rotatable knob with discrete or continuous thickness settings.

The volume rendering processor 162 projects the rendering region 210 onto an image portion 220 of an image plane(s) 222 (shown in FIG. 3). Following processing in the volume rendering processor 162, pixel data in the image portion 220 may be processed by the video processor 164 and then displayed on the display 166. The rendering region 210 may be located at any position and oriented at any direction within the volume 202. In some situations, depending on the size of the region being scanned, it may be advantageous for the rendering region 210 to be only a small portion of the volume 202.

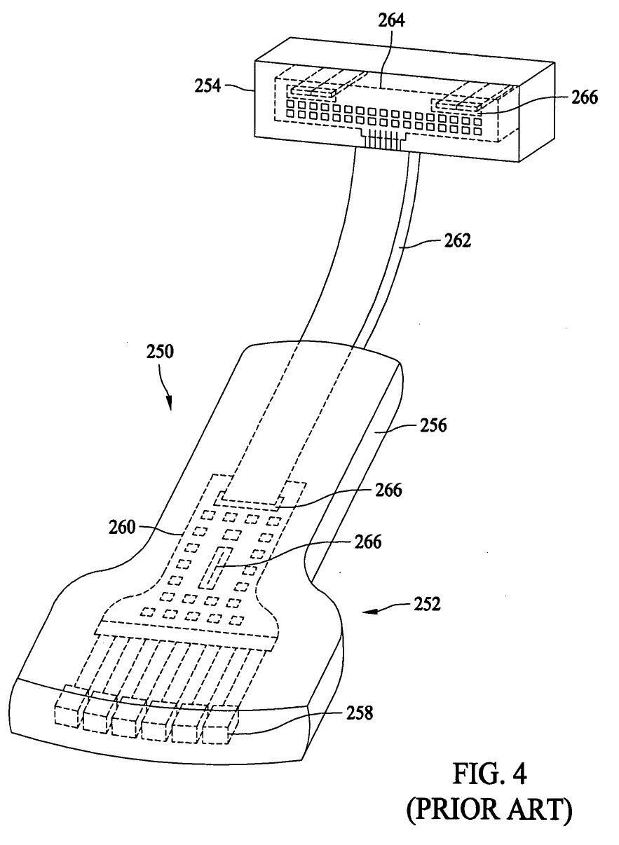

FIG. 4 illustrates a perspective view of a typical ultrasound probe 250 that generally includes a scan portion 252 and a connector 254. The scan portion 252 typically include a housing 256 having therein control components and operating components for performing ultrasound scans. For example, and in general, the housing 256 may include therein a transducer array 258 having a plurality of elements, such as, for example, piezoelectric elements (not shown) and control components, for example, passive electrical components, mounted to a printed circuit board 260. The printed circuit board 260 allows communication between the transducer array 258 and an ultrasound system (not shown) through a system cable 262, which is connected to the ultrasound system via the connector 254. Additionally, the connector 254 may include a printed circuit board 264 therein for connecting the system cable 262 to the ultrasound system.

In the ultrasound probe 250 the control components, and more particularly the passive electrical components, such as, for example, resistors, inductors, and capacitors, are surface mounted to a side of the printed circuit board 260 or 264. Additionally, the printed circuit boards 260 and 264 may include connection members 266 for providing interconnection within the ultrasound probe 250. For example, a connector 266 may be provided for interconnection of the system cable 262 to the printed circuit board 260, which printed circuit board 260 also provides connection to the transducer array 258.

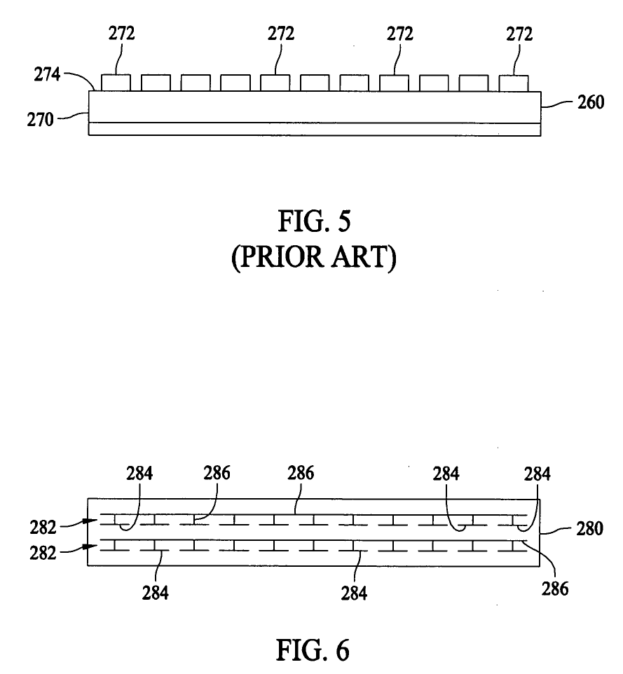

As shown in FIG. 5, the prior art printed circuit board 260, or the printed circuit board 264 includes a printed base 270 on which control components 272, such as, for example, resistors, inductors, and capacitors, are surface mounted to a top surface 274 thereof.

Various embodiments of the present invention provide a circuit board, for example, a printed circuit board for use in connection with an ultrasound probe having embedded passive electrical components therein. It should be noted that when reference is made herein to a circuit board or printed circuit board, this refers to any type of circuit board constructed of different materials including, for example, ceramic, glass, aluminum, metal bond, etc. Additionally, the circuit board may be of different configurations or formed of different substrates, including, for example, an LTCC substrate. Specifically, and as shown in FIG. 6, a printed circuit board 280 includes embedded therein (during the board fabrication process as is known), a plurality of control components for controlling the operation of the ultrasound probe. As shown in FIG. 6, a plurality of layers 282, which may be more than the two shown, include a plurality of control components embedded therein. As shown, the control components are a plurality of capacitors 284 connected together with traces 286. The printed circuit board 280 may be, for example, an interconnect board, multiplexing or control board within the housing or handle of an ultrasound probe used for controlling the activation of elements of a transducer array.

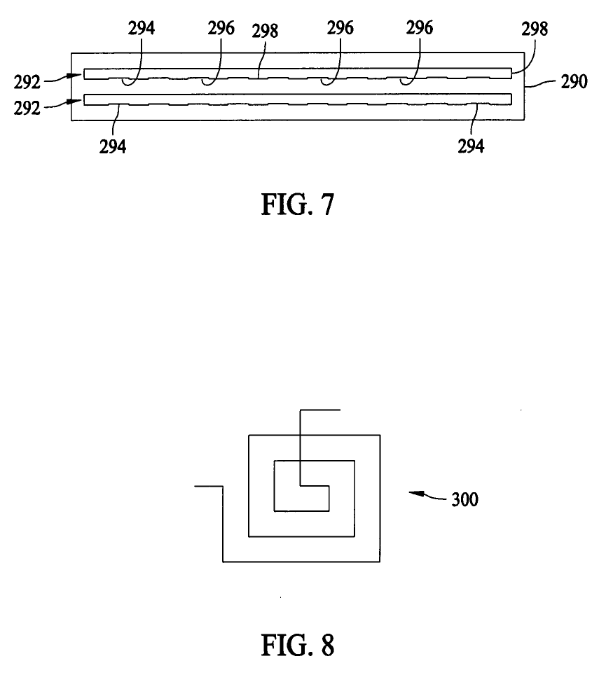

FIG. 7 shows another exemplary embodiment of a printed circuit board 290 having a plurality of layers 292 of control components. The control components may be resistors 294 or conductors 296 interconnected with traces 298. The printed circuit board 290 may be, for example, a tuning board for matching the impedance of elements of the transducer array to the ultrasound system in order to optimize the acoustic characteristics or parameters of the ultrasound system. It should be noted that one or more control components may be associated with a single element of the transducer array for controlling that element. Further, it should be noted that the various embodiments of the present invention are not limited to specific components, but may be implemented as a printed circuit board having passive electrical components of any kind embedded within the printed circuit board, such as, for example, any array of passive electrical components, such as a resistor array. Further, for example, the printed circuit boards may be any type of printed circuit board, as needed or desired for controlling the operation of the ultrasound system, including the ultrasound probe, such as, for filtering transmit and receive signals within the system.

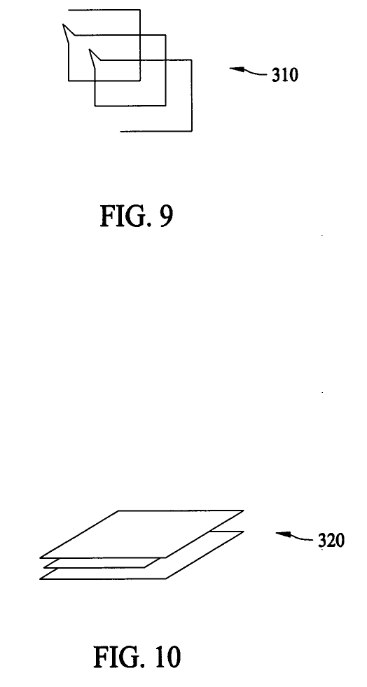

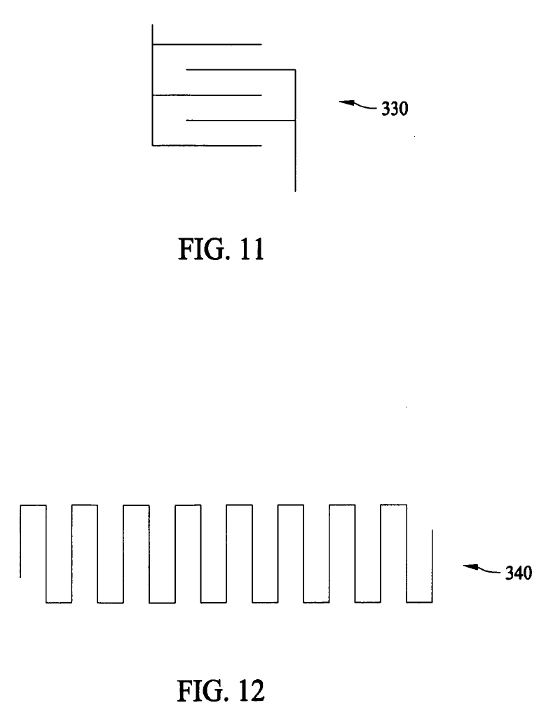

Further, and for example, different configurations of passive electrical components may be implemented. For example, and without limitation, FIG. 8 shows a spiral inductor 300 which may be implemented in the various embodiments of the present invention. FIG. 9 shows a helical inductor 310 which may be embedded in a printed circuit board according to the various embodiments of the present invention. FIG. 10 shows a parallel plate capacitor 320 that may be embedded within a printed circuit board according to the various embodiments of the present invention. FIG. 11 shows another capacitor 330 that may be embedded within a printed circuit board according to the various embodiments of the present invention. FIG. 12 shows a resistor 340 that may be embedded within a printed circuit board according to various embodiments of the present invention.

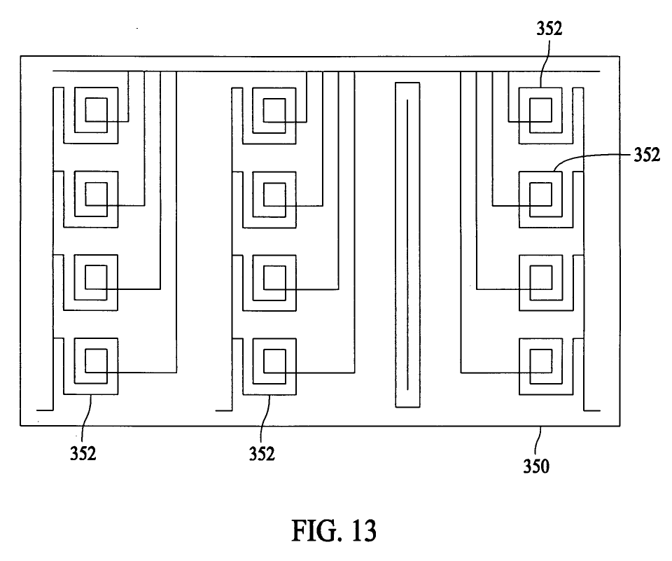

Thus, various embodiments of the present invention provide for embedding impassive electrical components within a printed circuit board for use in connection with an ultrasound system, and more particularly for use in connection with an ultrasound probe of the ultrasound system. As shown in FIG. 13, a printed circuit board 350 may include a plurality of embedded electrical passive components 352, shown as inductors, for use in a probe. For example, the printed circuit board 350 may be configured as a tuning board for a connector of the ultrasound probe. However, it should be noted that the type of passive electrical components, and the type of board thereby configured, may be modified as desired or needed based upon the particular application, type of probe, type of ultrasound system, or otherwise. Further, the printed circuit board 350 may be positioned in any location within the ultrasound probe, and is not limited to the probe handle, probe connector, or otherwise.

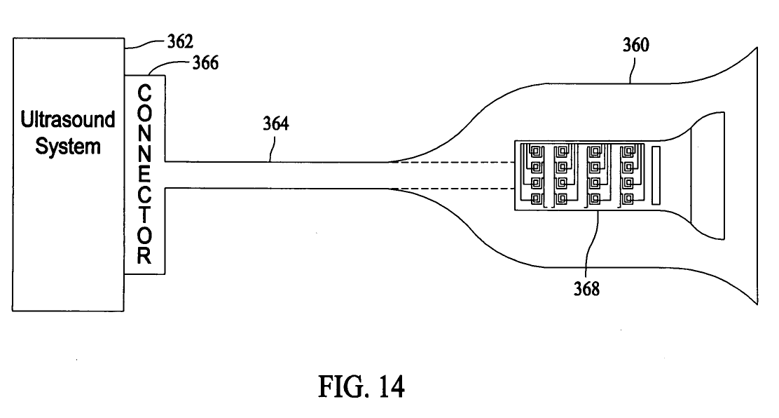

Thus, as shown in FIG. 14, the various embodiments of the present invention provide a ultrasound probe 360 connected to an ultrasound system 362 via a system cable 364 and having a connector 366 for connecting the ultrasound probe 360 to the ultrasound system 362. The ultrasound probe 360 includes various components, including, a printed circuit board 368 having embedded passive electrical components as described herein. Thus, various embodiments of the present invention provide a printed circuit board having control components embedded therein, more particularly, passive electrical components embedded therein for use in connection with an ultrasound probe.

While the invention has been described in terms of very specific embodiments, those skilled in the art will recognize that the invention can be practiced with modification within the spirit and scope of the claims.

What is claimed is:

1. An ultrasound probe comprising:
a scan portion for scanning an object;
a connector for connecting the scan portion to an ultrasound system; and
at least one printed circuit board within at least one of the scan portion and the connector, the at least one printed circuit board having embedded passive components.

2. An ultrasound probe in accordance with claim 1 further comprising a system cable connecting the scan portion to the connector.

3. An ultrasound probe in accordance with claim 1 wherein the passive components comprise at least one of (i) resistors, (ii) inductors and (iii) capacitors.

4. An ultrasound probe in accordance with claim 1 wherein the at least one printed circuit board comprises a tuning board configured to provide impedance matching of transducer elements in the scan portion with a system output of the ultrasound system.

5. An ultrasound probe in accordance with claim 4 wherein the tuning board comprises a plurality of inductors.

6. An ultrasound probe in accordance with claim 1 wherein the at least one printed circuit board comprises a multiplexing board configured to provide multiplexing operation to control transducer elements in the scan portion.

7. An ultrasound probe in accordance with claim 1 wherein the at least one printed circuit board comprises an interconnect board configured to provide transmission of signals between transducers elements within the scan portion and the ultrasound system.

8. An ultrasound probe in accordance with claim 1 wherein the at least one printed circuit board comprises a filter board configured to filter transmit and receive signals.

9. An ultrasound probe in accordance with claim 1 wherein the at least one printed circuit board comprises at least one array of passive components.

10. An ultrasound probe in accordance with claim 1 wherein the scan portion is configured to perform medical imaging in combination with the ultrasound system.

11. An ultrasound probe in accordance with claim 1 wherein at least one printed circuit board is provided within the scan portion and at least one printed circuit board is provided within the connector.

12. An ultrasound probe in accordance with claim 1 wherein the at least one printed circuit board comprises integrated circuits having passive components therein and the integrated circuits are embedded within the printed circuit board.

13. A circuit board for an ultrasound probe, said circuit board comprising:
a plurality of embedded passive electrical components configured to allow positioning within at least one of a scan portion and a connector of an ultrasound probe, the passive components including at least one of resistors, inductors and capacitors; and
at least one connector to provide interconnection and communication with an ultrasound system controlling the ultrasound probe.

14. A circuit board in accordance with claim 13 wherein the at least one connector is configured for connection to a system cable of the ultrasound system.

15. A circuit board in accordance with claim 13 wherein the at least one connector is configured for connection to a plurality of transducer elements within a probe of the ultrasound system.

16. A circuit board in accordance with claim 13 wherein the passive electrical components are configured to tune the ultrasound probe to provide impedance matching of transducer elements in the scan portion with a system output of the ultrasound system.

17. A circuit board in accordance with claim 13 wherein the passive electrical components are configured to provide multiplexing operation to control transducer elements in the scan portion.

18. A circuit board in accordance with claim 13 wherein each of the passive electrical components are connected to each of a transducer element of a transducer array in the scan portion.

19. A method for controlling operation of an ultrasound probe, said method comprising:
embedding passive electrical components in a printed circuit board for connection within an ultrasound probe; and
configuring the embedded passive electrical components to communicate with an ultrasound system for controlling the ultrasound probe.

20. A method in accordance with claim 19 further comprising configuring the passive electrical components to provide one of a (i) tuning, (ii) multiplexing and (iii) filtering operation.
